(19) United States
(12) Patent Application Publication
(10) Pub. No.: US 2013/0197365 A1
(43) Pub. Date: Aug. 1, 2013

(54) ULTRASONIC DIAGNOSTIC APPARATUS AND ULTRASONIC DIAGNOSTIC APPARATUS CONTROL METHOD

(71) Applicants: Kabushiki Kaisha Toshiba, Tokyo (JP); Toshiba Medical Systems Corporation, Otawara-shi (JP)

(72) Inventor: Tatsuro BABA, Otawara-shi (JP)

(73) Assignees: Toshiba Medical Systems Corporation, Otawara-shi (JP); Kabushiki Kaisha Toshiba, Tokyo (JP)

(21) Appl. No.: 13/755,160

(22) Filed: Jan. 31, 2013

(30) Foreign Application Priority Data
Jan. 31, 2012 (JP) 2012-018844
Oct. 26, 2012 (JP) 2012-236554

(51) Int. Cl. A61B 8/14; A61B 8/08; A61B 8/06; A61B 8/00

(52) U.S. Cl. CPC A61B 8/145; A61B 8/4488; A61B 8/5207; A61B 8/06; A61B 8/5223; A61B 8/4444; A61B 8/483; A61B 8/463; A61B 8/488; A61B 8/469; A61B 8/466; A61B 8/54; A61B 8/565
USPC 600/441; 600/447

(57) ABSTRACT

In one embodiment, an ultrasonic diagnostic apparatus continuously generates driving signals by frequency-modulating waveforms having a plurality of center frequencies respectively assigned to orientation directions and multiplexing the waveforms and transmits continuous waves, and generates beam signals corresponding to the respective orientation directions by adding the respective echo signals and demultiplexing the signals for the respective center frequencies, demodulates beam signals corresponding to the respective orientation directions, frequency-analyzes the demodulated beam signals, calculates two-dimensional (beam direction and range direction) mapping of beam signals.

### CROSS-REFERENCE TO RELATED APPLICATIONS

This application is based upon and claims the benefit of priority from Japanese Patent Applications No. 2012-018844, filed Jan. 31, 2012; and No. 2012-236554, filed Oct. 26, 2012, the entire contents of all of which are incorporated herein by reference.

### FIELD

Embodiments described herein relate generally to an ultrasonic diagnostic apparatus which can perform simultaneous measurement in orientation directions when executing CWD (Continuous Wave Doppler) measurement using CWs (Continuous Waves), and a method of controlling the apparatus.

### BACKGROUND

An ultrasonic diagnostic apparatus emits ultrasonic pulses generated by transducers provided in an ultrasonic probe into an object to be examined, and receives reflected ultrasonic waves generated by differences in acoustic impedance of the tissues of the object via the transducers, thereby acquiring biological information. This apparatus can perform real-time display of image data by the simple operation of bringing the ultrasonic probe into contact with the surface of the body and allows the observation of a moving object such as the heart, and hence is widely used for morphological diagnosis and functional diagnosis of circulatory organ regions and various organs.

Such ultrasonic diagnostic apparatuses use a blood flow velocity measurement method called a CWD method when performing ultrasonic diagnosis. This method is designed to measure a blood flow velocity by performing Doppler imaging using continuous ultrasonic waves. The method is generally used to measure a high-speed blood flow in a deep region.

### BRIEF DESCRIPTION OF THE DRAWINGS

FIG. 1 is a block diagram of an ultrasonic diagnostic apparatus 1 according to an embodiment;

FIG. 2 is a view for explaining a simultaneous multidirectional CWD function;

FIG. 3 is a graph showing an example of voltage waveforms assigned to three different orientation directions;

FIG. 4 is a graph showing a multiplex wave of the three voltage waveforms shown in FIG. 3;

FIG. 5 is a graph showing the spectrum distribution of the reception beam obtained by transmitting the multiplex wave shown in FIG. 4;

FIG. 6 is a graph showing the spectrum distribution obtained by demultiplexing the reception beam obtained by multiplex transmission by using a bandpass filter;

FIG. 7 is a view showing an example in which different frequencies are respectively assigned to 13 different orientation directions at 0.05 MHz intervals, with a frequency of 2.0 MHz being assigned to the center of the deflected beam;

FIG. 8 is a view for explaining a conventional CDW method;

FIG. 9 is a view for explaining application example 1 of a simultaneous multidirectional CWD function;

FIG. 10 is a view for explaining application example 1 of the simultaneous multidirectional CWD function;

FIG. 11 is a view for explaining application example 2 of the simultaneous multidirectional CWD function;

FIG. 12 is a view for explaining application example 2 of the simultaneous multidirectional CWD function;

FIG. 13 is a view for explaining application example 2 of the simultaneous multidirectional CWD function;

FIG. 14 is a view for explaining application example 3 of the simultaneous multidirectional CWD function (conventional CWD);

FIG. 15 is a view for explaining application example 3 of the simultaneous multidirectional CWD function (multi-beams);

FIG. 16 is a view for explaining application example 3 of the simultaneous multidirectional CWD function (spectrum distributions);

FIG. 17 is a view for explaining application example 3 of the simultaneous directional CWD function (blood flow velocity distribution);

FIG. 18 is a view for explaining application example 4 of the simultaneous directional CWD function (two-dimensional system);

FIG. 19 is a view for explaining application example 4 of the simultaneous directional CWD function (three-dimensional system);

FIG. 20 is a view for explaining application example 5 of the simultaneous directional CWD function (end-fire two-dimensional phased array system);

FIG. 21 is a view for explaining application example 5 of the simultaneous directional CWD function (Concentric circle-like frequency distribution);

FIG. 22 is a view for explaining application example 5 of the simultaneous directional CWD function (end-fire probe system);

FIG. 23 is a view for explaining application example 6 of the simultaneous directional CWD function (two-beams simultaneous measurement of carotid artery);

FIG. 24 is a view for explaining application example 6 of the simultaneous directional CWD function (time difference of two-beams simultaneous measurement);

FIG. 25 is a view for explaining application example 6 of the simultaneous directional CWD function (two-beams simultaneous measurement by sector probe);

FIG. 26A is a block diagram of an ultrasonic diagnostic apparatus 1 according to a second embodiment;

FIG. 26B is a block diagram showing the arrangement of an ultrasonic transmission unit 21 which implements a frequency-divided FMCWD function;

FIG. 27 is a view for explaining transmission processing based on the frequency-divided FMCWD function (divided power spectra);

FIG. 28 is a view for explaining transmission processing based on the frequency-divided FMCWD function (time-domain chirp waveforms combining);

FIG. 29 is a view for explaining Tx and Rx beams based on the frequency-divided FMCWD function;

FIG. 30 is a block diagram showing the arrangement of an ultrasonic reception unit 22 which implements the frequency-divided FMCWD function;

FIG. 31 is a view for explaining reception processing based on the frequency-divided FMCWD function (frequency analysis);

FIG. 32 is a conceptual view for explaining demodulation processing according to this application example;

FIG. 33A is a graph for explaining the effects of demodulation processing according to the application example (Rx waveforms);

FIG. 33B is a graph for explaining the effects of demodulation processing according to the application example (Rx spectra); and

FIG. 34 is a graph for explaining the effects of demodulation processing according to the application example.

### DETAILED DESCRIPTION

In general, according to one embodiment, an ultrasonic diagnostic apparatus includes a transmission unit, a reception unit, a frequency analyzing unit and an image generation unit. The transmission unit continuously generates driving signals by frequency-modulating a plurality of waveforms having a plurality of center frequencies respectively assigned to a plurality of orientation directions and multiplexing the plurality of waveforms and transmit continuous waves deflected from a perpendicular direction to an array plane of ultrasonic transducers of an ultrasonic probe via an ultrasonic probe by supplying the driving signals to the ultrasonic transducers with different delay times. The reception unit generates a plurality of beam signals corresponding to the respective orientation directions by adding the respective echo signals received by the respective ultrasonic transducers with different delay times for the respective ultrasonic transducers and demultiplexing the signals for the respective center frequencies and demodulate a plurality of beam signals corresponding to the respective orientation directions, frequency-analyzes the plurality of demodulated beam signals, and calculates beam signals including distance information concerning a depth direction in each orientation direction. The frequency analyzing unit detects shift frequency spectrums for the respective orientation directions by using a plurality of beam signals including distance information concerning the respective orientation directions. The image generation unit generates an ultrasonic image based on the shift frequency spectrums concerning the depth direction in the each orientation direction.

The embodiment will be described below with reference to the accompanying drawing. Note that the same reference numerals in the following description denote constituent elements having almost the same functions and arrangements, and a repetitive description will be made only when required.

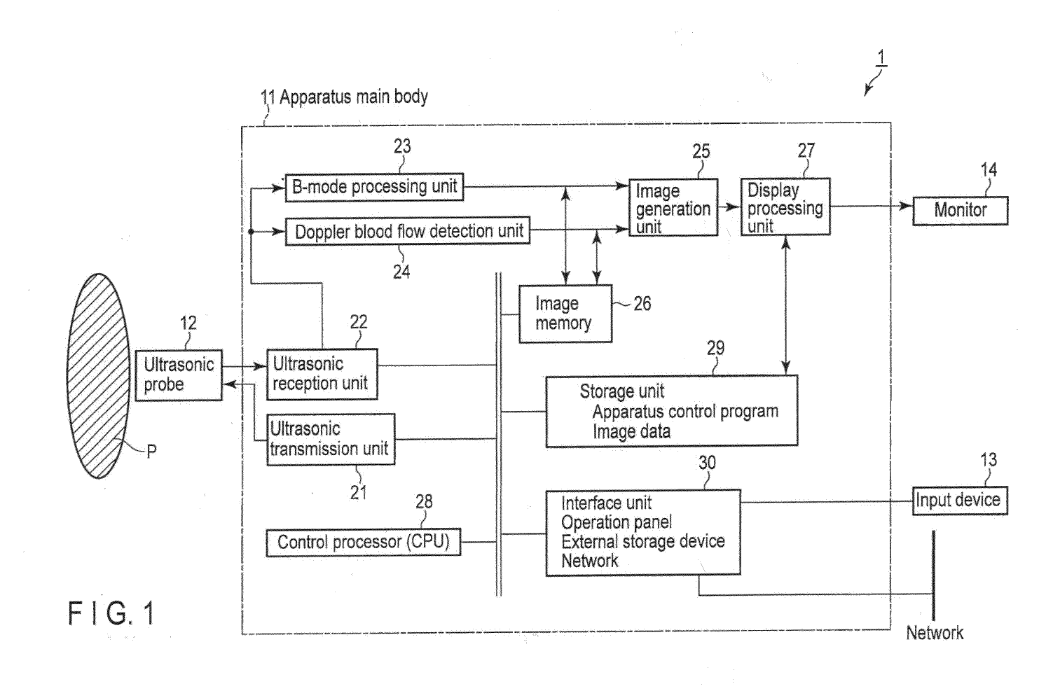

FIG. 1 is a block diagram showing the arrangement of an ultrasonic diagnostic apparatus 1 according to this embodiment. As shown in FIG. 1, the ultrasonic diagnostic apparatus 1 includes an ultrasonic probe 12, an input device 13, a monitor 14, an ultrasonic transmission unit 21, an ultrasonic reception unit 22, a B-mode processing unit 23, a Doppler blood flow detection unit 24, an image generation unit 25, an image memory 26, a display processing unit 27, a control processor (CPU) 28, a storage unit 29, and an interface unit 30. The function of each constituent element will be described below.

The ultrasonic probe 12 is a device (probe) which transmits ultrasonic waves town object, and receives reflected waves from the object based on the transmitted ultrasonic waves. The ultrasonic probe 12 has, on its distal end, a plurality of ultrasonic transducers, a matching layer, a backing member, and the like. The ultrasonic transducers transmit ultrasonic waves in a desired direction in a scan area based on driving signals from the ultrasonic transmission unit 21, and convert reflected waves from the object into electrical signals. The matching layer is an intermediate layer which is provided for the ultrasonic transducers to make ultrasonic energy efficiently propagate. The backing member prevents ultrasonic waves from propagating backward from the ultrasonic transducers. When the ultrasonic probe 12 transmits an ultrasonic wave to an object P, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by a moving blood flow is subjected to a frequency shift depending on the velocity component of the moving body in the ultrasonic transmission/reception direction due to the Doppler effect.

Note that the ultrasonic probe 12 has a band which allows CWD transmission/reception. In addition, this probe may be a one-dimensional array probe having a plurality of ultrasonic transducers arrayed one-dimensionally or a two-dimensional array probe having a plurality of ultrasonic transducers arrayed two-dimensionally.

The input device 13 is connected to an apparatus main body 11 and includes various types of switches, buttons, a trackball, a mouse, and a keyboard which are used to input, to the apparatus main body 11, various types of instructions, conditions, an instruction to set a region of interest (ROI), various types of image quality condition setting instructions, and the like from an operator.

The monitor 14 displays morphological information and blood flow information in the living body, Doppler waveforms in the respective orientation directions, and the like based on video signals from the display processing unit 27.

The ultrasonic transmission unit 21 includes an oscillation generation unit, transmission frequency dividing unit, and transmission driver (none of which are shown). The oscillation generation unit repeatedly generates oscillation waveforms having a predetermined frequency fr Hz (period: 1/fr sec). The transmission frequency dividing unit frequency-divides oscillation waveforms from the oscillation generation unit to generate waveforms having desired frequencies. The transmission driver supplies multiplex waves obtained by combining a plurality of waveforms corresponding to different frequencies generated by frequency division processing to the respective ultrasonic transducers with predetermined delay times.

The ultrasonic reception unit 22 includes an amplifier circuit, A/D converter, reception delay unit, and adder (none of which are shown). The amplifier circuit amplifies an echo signal received via the probe 12 for each channel. The A/D converter converts each amplified analog echo signal into a digital echo signal. The delay circuit gives the digitally converted echo signals delay times necessary to determine reception directivity and perform reception focusing. The adder then performs addition processing for the signals.

The B-mode processing unit 23 receives an echo signal from the reception unit 22, and performs logarithmic amplification, envelope detection processing, and the like for the signal to generate data whose signal intensity is expressed by a luminance level.

The Doppler blood flow detection unit 24 obtains Doppler waveforms and blood flow information such as an average velocity, variance, or power as blood flow data by extracting and analyzing blood flow signals from the echo signals received from the ultrasonic reception unit 22. The Doppler blood flow detection unit 24 also obtains Doppler waveforms in the respective orientation directions and blood flow information such as an average velocity, variance, or power as blood flow data by detecting Doppler shift frequencies in the respective orientation directions in accordance with the simultaneous multidirectional CWD function (to be described later).

The image generation unit 25 generates two-dimensional or three-dimensional image data by executing RAW-pixel conversion (or voxel conversion) for the two-dimensional or three-dimensional RAW data received from the B-mode processing unit 23 and the image memory 26. The image generation unit 25 performs predetermined image processing such as volume rendering, MPR (Multi Planar Reconstruction) or MIP (Maximum Intensity Projection) for the generated image data.

The image memory 26 generates two-dimensional or three-dimensional B-mode RAW data by using a plurality of B-mode data received from, for example, the B-mode processing unit 23.

The display processing unit 27 executes various kinds of adjustments concerning dynamic range, brightness, contrast, γ curve correction, RGB conversion, and the like for various kinds of image data generated and processed by the image generation unit 25.

The control processor 28 has the function of an information processing apparatus (computer) and controls the operation of this ultrasonic diagnostic apparatus main body. The control processor 28 reads out a control program for implementing the simultaneous multidirectional CWD function (to be described later) from the storage unit 29, expands the program in its own memory, and executes control concerning simultaneous multidirectional CWD and calculations (calculations of a compound, the spatial distribution of signal intensities, automatic angle correction, the intravascular distribution of blood flow velocities, and diagnostic index values) using the Doppler signals in the respective orientation directions which are obtained by the simultaneous multidirectional CWD function.

The storage unit 29 stores the control program for implementing the simultaneous multidirectional CWD function (to be described later), diagnosis information (patient ID, findings by doctors, and the like), a diagnostic protocol, transmission/reception conditions, a program for implementing a speckle removal function, a body mark generation program, a conversion table for setting in advance a color data range used for visualization for each diagnostic region, and other data groups. The storage unit 29 is also used to store images in the image memory (not shown), as needed. It is possible to transfer data in the storage unit 29 to an external peripheral device via the interface unit 30.

The interface unit 30 is an interface associated with the input device 13, a network, and a new external storage device (not shown). The interface unit 30 can transfer, via a network, data such as ultrasonic images, analysis results, and the like obtained by this apparatus to another apparatus.

### (Simultaneous Multidirectional CWD Function)

The simultaneous multidirectional CWD function of the ultrasonic diagnostic apparatus 1 will be described next. When performing blood flow measurement using the CWD method, this function transmits multiplex waves to which different frequencies are respectively assigned to the orientation directions of ultrasonic beams from the respective ultrasonic transducers and detects the Doppler shift frequencies of the respective frequencies from the reflected waves obtained by the multiplex waves, thereby simultaneously executing CDW in the respective orientation directions.

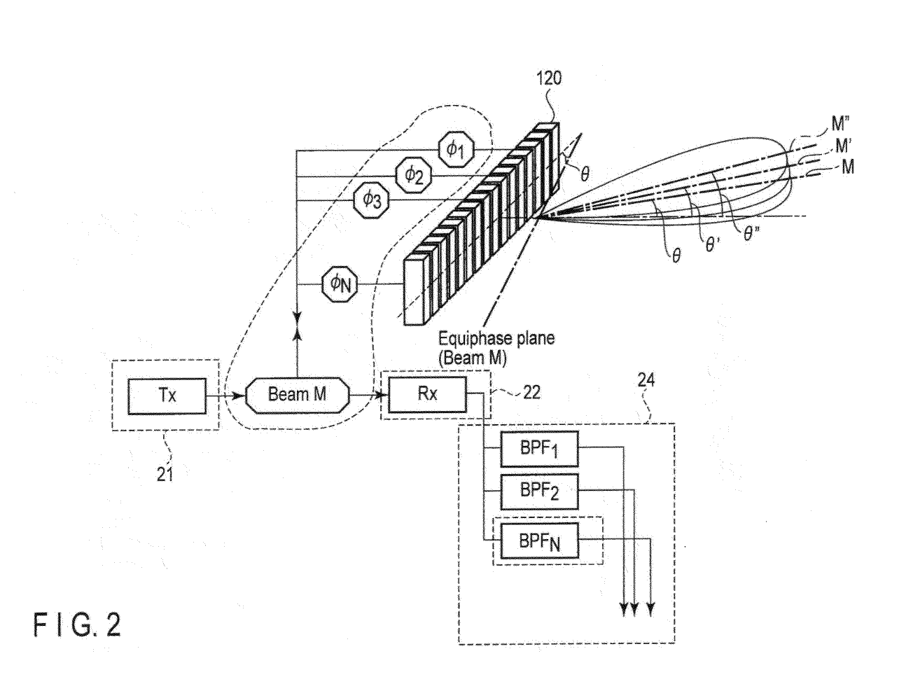

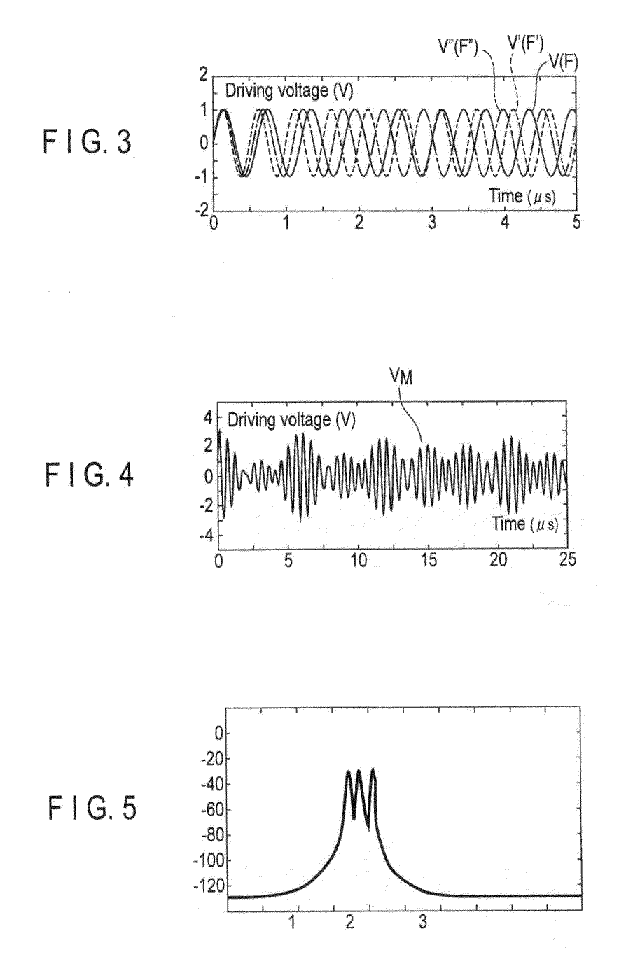

FIG. 2 is a view for explaining the simultaneous multidirectional CWD function. For the sake of simplicity, assume that this function simultaneously performs CWD measurement in three directions.

Referring to FIG. 2, for example, frequencies F, F′, and F″ are respectively assigned to orientation directions θ, θ′, and θ″. In this case, the ultrasonic transmission unit 21 frequency-divides oscillation waveforms to generate a driving voltage waveform V(F) assigned to the orientation direction θ, a driving voltage waveform V′(F′) assigned to the orientation direction θ′, and a driving voltage waveform V″(F″) assigned to the orientation direction θ″. The ultrasonic transmission unit 21 generates a multiplex wave V_{M} like that shown in FIG. 4 by combining (multiplexing) the generated waveforms V(F), V′(F′), and V″(F″), and supplies the wave as a driving signal having a phase delay (φ₁, φ₂, φ₃, . . . , φ_{N}) for each ultrasonic transducer to each corresponding ultrasonic transducer, as shown in FIG. 2. As a result, the ultrasonic probe 12 transmits a beam M corresponding to the orientation direction θ, a beam M′ corresponding to the orientation direction θ′, and a beam M″ corresponding to the orientation direction θ″. Note that FIG. 2 exemplifies only an equiphase plane of the transmission beam M corresponding to the orientation direction θ.

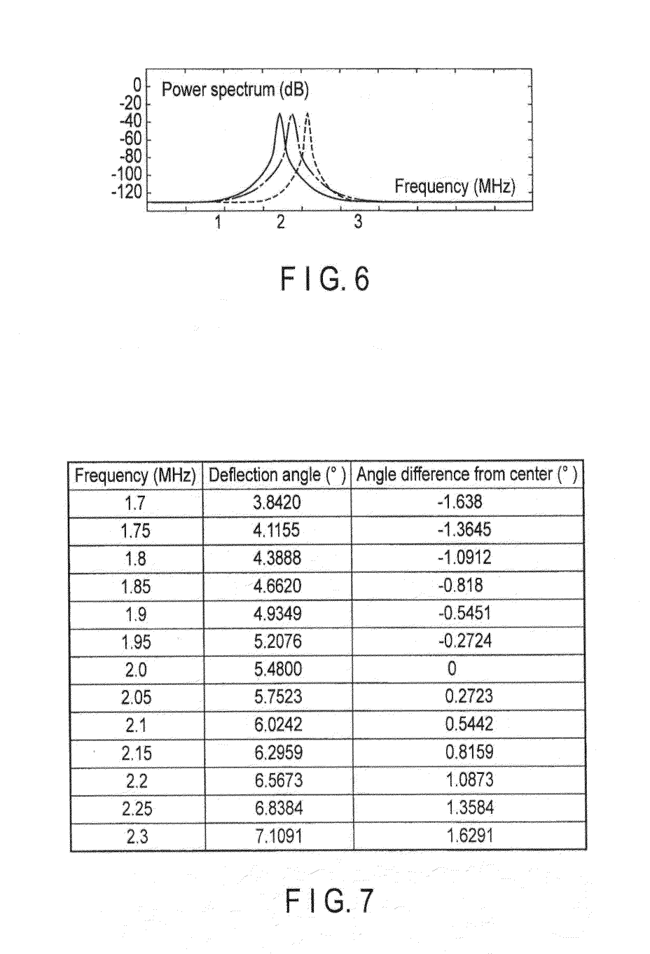

Multiplex waves respectively having predetermined phase delays are transmitted for the respective ultrasonic transducers. The transmission multiplex waves are reflected by the inside of the object body are received as reflected waves by the respective ultrasonic transducers. The ultrasonic reception unit 22 generates a reception beam by amplifying the respective reflected waves received by the respective ultrasonic transducers and adding them with delays. This reception beam originates from the transmission multiplex waves obtained by multiplexing three waveforms in different frequency bands, and hence has a spectrum waveform like that shown in, for example, FIG. 5. The Doppler blood flow detection unit 24 demultiplexes this beam into echo signals having spectra corresponding to the respective orientation directions like those shown in FIG. 6, and executes Doppler measurement processing for each echo signal.

The above description has exemplified the case in which the apparatus simultaneously performs CWD measurement upon assigning different frequencies to the three directions, namely the orientation directions θ, θ′, and θ″. However, CWD measurement is not limited to this, and it is however possible to simultaneously perform CWD measurement by the same processing upon assigning different frequencies to n orientation directions (where n is an arbitrary number equal to or more than 2). Note that FIG. 7 shows an example in which different frequencies are respectively assigned to 13 different orientation directions at 0.05 MHz intervals, with a frequency of 2.0 MHz being assigned to an orientation direction (The central deflection angle θ).

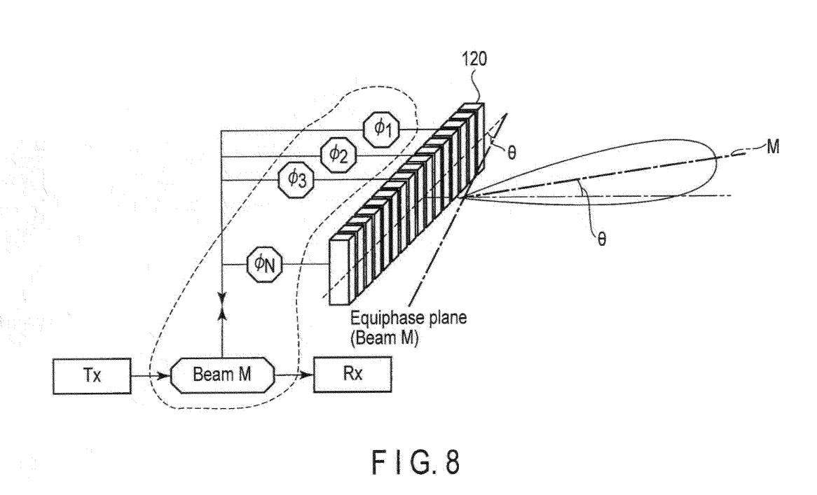

The above simultaneous multidirectional CWD function has not existed. Conventionally, for example, as shown in FIG. 8, beam forming in one orientation direction θ is performed by applying, to each ultrasonic transducer, a driving voltage with a predetermined frequency F phase-delayed for each ultrasonic transducer. In contrast to this, as shown in FIG. 2, this simultaneous multidirectional CWD function supplies multiplex waves with different frequencies assigned to the respective orientation directions of ultrasonic beams to the respective ultrasonic transducers while phase-delaying the waves for the respective ultrasonic transducers, and detects the Doppler shift frequencies of the respective frequencies from the reflected waves obtained by the multiplex waves. This makes it possible to simultaneously execute CDW in the respective orientation directions.

### Application Example 1

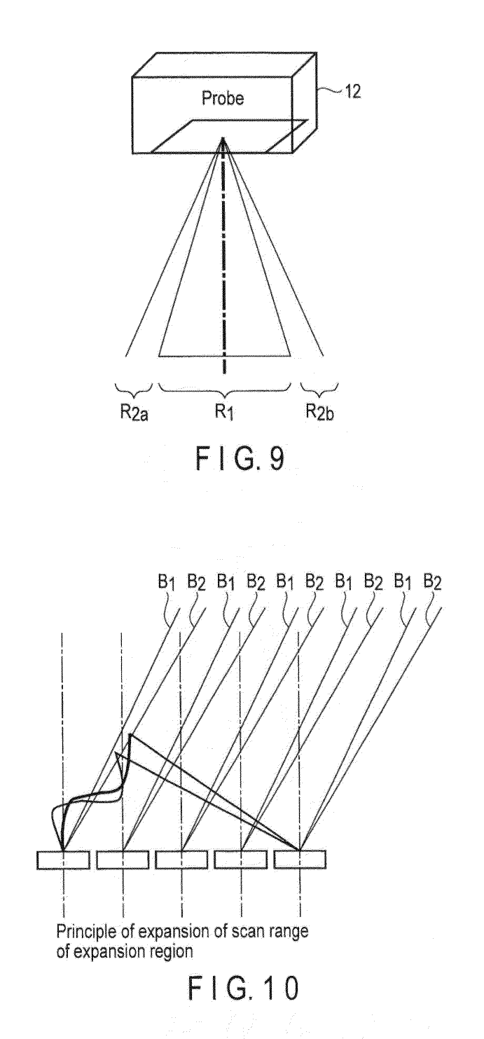

According to conventional CWD, as shown in FIG. 10, a beam B1 is deflected in a conventional steering region R1 shown in FIG. 9 by phase-delaying a single frequency. This is because since the time delay is not used unlike PWD, some restriction is imposed on the deflection range of deflection angels, and a very narrow reception aperture must be used. For this reason, deflection in a range exceeding 2θ causes aliasing, and hence some restriction is imposed on the deflection range of the beam direction B1. Although an increase in aperture trades off with artifacts, it is necessary to perform aperture control such as weighting.

In application example 1, the simultaneous multidirectional CWD function eliminates the above restrictions and increases the steering angle of a beam. That is, the simultaneous multidirectional CWD function according this application example phase-delays a beam direction B2 having a frequency different from that of the beam direction B1 in FIG. 10 in expansion regions R2a and R2b in FIG. 9. This can further ensure a margin corresponding to a phase of 2θ in the expansion regions R2a and R2b.

More specifically, assume that 2-MHz driving is performed in a conventional steering range, and the conventional steering range is expanded outward. In this case, deflection delay data at 2 MHz in the conventional steering range is fixed, and the driving frequency to be assigned to each orientation direction in the expanded steering range is increased to 2 MHz to 2.4 MHz. This makes it possible to expand the steering range to about 14° when the one-side deflection upper limit is 10° in the related art. At the time of reception, reception is delayed in synchronism with frequency. This allows the simultaneous multidirectional CWD function to ensure a wider steering range than the related art.

Note that at the time of strong deflection, it is necessary to reduce the aperture by apodization as in the related art. However, the influence of this operation is considered low, and hence the operation can be used to reduce the sensitivity deterioration at an end portion. The above description has exemplified the case in which beam steering based on the simultaneous multidirectional CWD function expands the deflection range by the expanded regions R2a and R2b relative to the conventional steering range R1, as shown in FIGS. 9 and 10. However, the expanded ranges are not limited to only the expanded regions R2a and R2b, and it is possible to further extend the deflection limit by assigning sequentially dropped frequencies to the further expanded regions.

### Application Example 2

Application example 2 is designed to improve the blood flow measurement accuracy by expanding the simultaneous measurement range by using the simultaneous multidirectional CWD function.

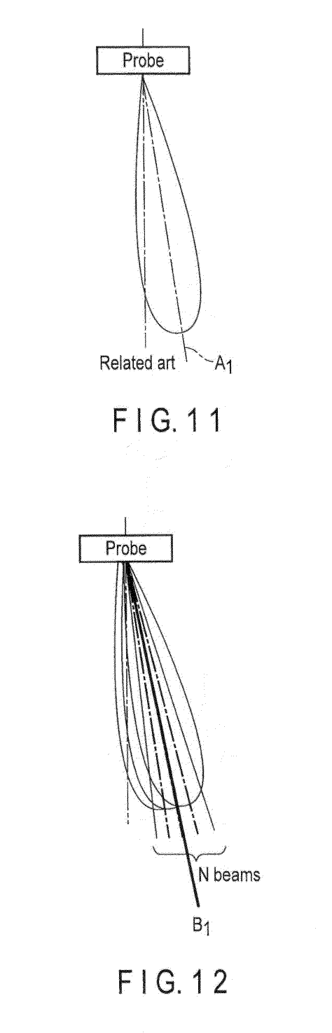

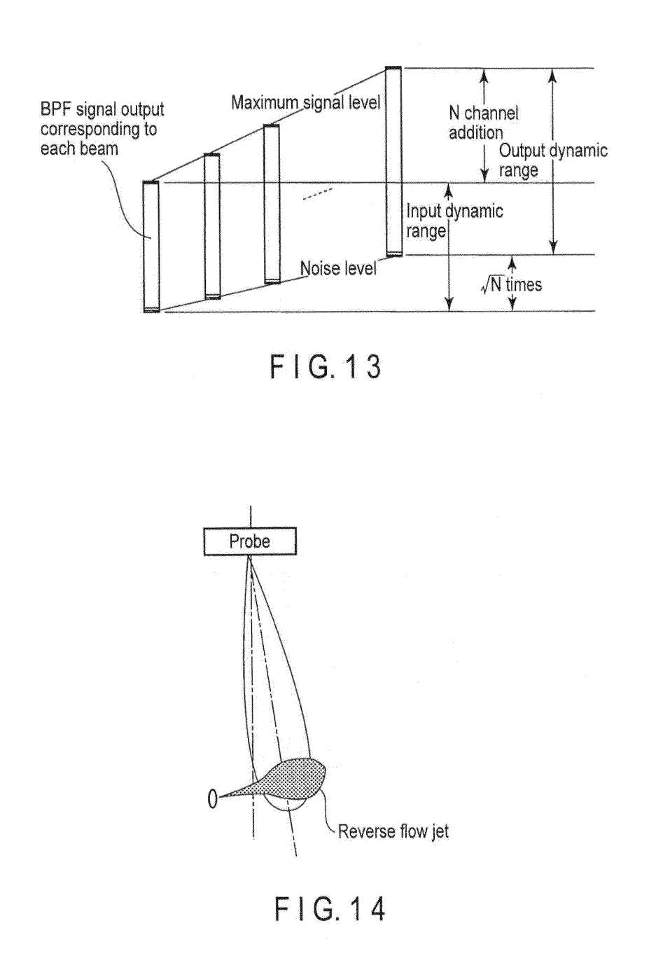

FIGS. 11, 12, and 13 are views for explaining application example 2 of the simultaneous multidirectional CWD function. As shown in FIG. 11, in the related art, an acoustic field is formed centered on a main beam axis A1. In this case, the simultaneous measurement range depends on expansion amount control on a beam shape (acoustic field) based on aperture control (e.g., expanding a beam by reducing the aperture and focusing at a far distance). In contrast to this, the simultaneous multidirectional CWD function superimposes a plurality of beam acoustic fields (N beam acoustic fields) centered on an axis B1 of a central beam, as shown in FIG. 12. This function extracts a signal by detecting each echo signal obtained from N beam acoustic fields by using different bandpass filters for the respective beams as shown in FIG. 13, and compounds the obtained beam information (acquires an ensemble average). Increasing the information obtained by using N beams in this manner can increase the S/N ratio by N^{1/2}.

### Application Example 3

Application example 3 is designed to grasp, for example, the shape of a reverse flow jet in the cardiac cavity by the simultaneous multidirectional CWD function.

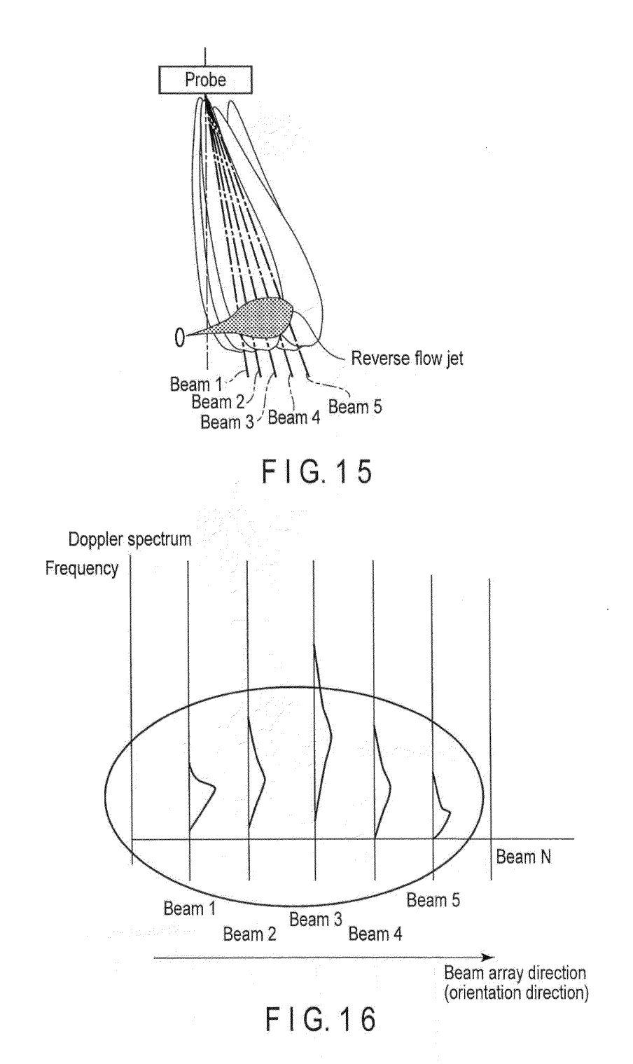

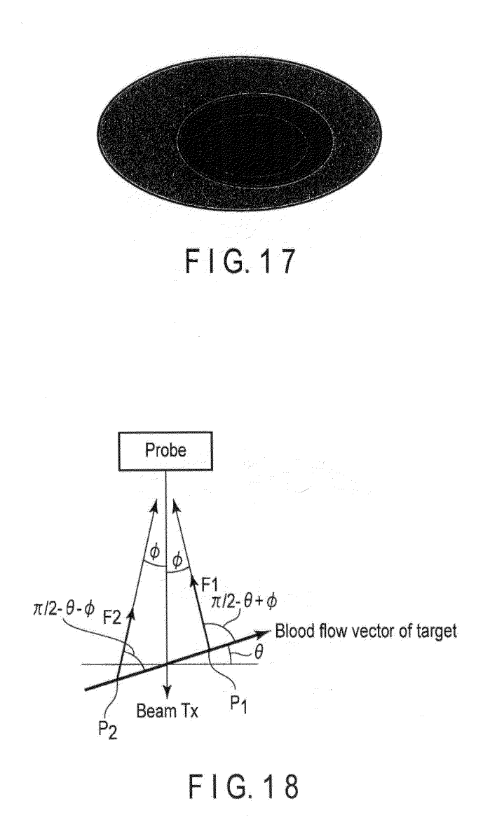

FIGS. 14, 15, 16, and 17 are views for explaining application example 3 of the simultaneous multidirectional CWD function. As shown in FIG. 14, conventional CWD measurement can only obtain blood flow information dependent on a beam profile, and hence can only capture a Doppler shift component as a volume total. In contrast to this, the simultaneous multidirectional CWD function superimposes N beams as shown in FIG. 15, separates the beams for the respective frequency bands, and detects the spectra of Doppler shift frequencies in the respective frequency bands as distributions in the beam array directions (orientation directions) as shown in, for example, FIG. 16. This makes it possible to measure blood flow information (a maximum value, power value, and the like) for each beam. In addition, these results allow to visually estimate the distributions of maximum velocities or power values in the beam array directions (orientation directions) by generating a color map (FIG. 17) with colors being assigned in accordance with the maximum velocities or power etc. Furthermore, such distributions of maximum velocities and the like allow to grasp up to which orientation direction a reverse flow jet like that shown in FIG. 15 exerts influence (i.e., a quantitative distribution of a reverse flow jet).

### Application Example 4

Application example 4 is designed to automatically correct a transmission angle by the simultaneous multidirectional CWD function. Note that a conventional angle correction algorithm is described in detail in, for example, Jpn. Pat. Appln. KOKAI Publication No. 2008-301892.

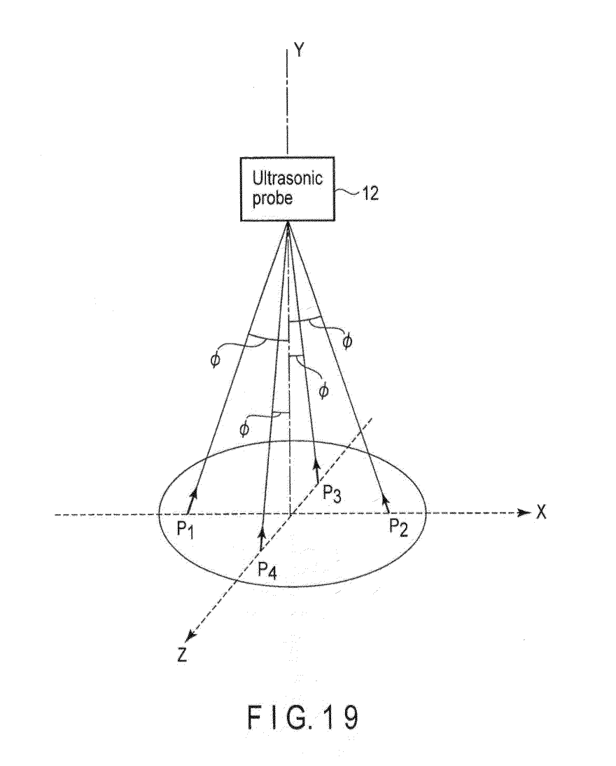

FIGS. 18 and 19 are views for explaining application example 4 of the simultaneous multidirectional CWD function. An example of transmission angle correction on a two-dimensional section will be described first with reference to FIG. 18. As shown in FIG. 18, assume that it is possible to simultaneously measure blood flow velocities from two directions on a two-dimensional section (in this case, a velocity or frequency from a point P1 and a velocity or frequency from a point P2), and a beam angle φ and angle 2φ between beams are known. In this case, a true blood flow velocity f0 can be calculated as follows.

First of all, a frequency f1 from the point P1 and a frequency f2 from the point P2 can be expressed as follows by using f0, φ, and θ, with θ representing the direction angle of a blood flow vector of a target:

f2=f0·sin(θ/2−θ−φ) (1)

f1=f0·sin(θ/2−θ−φ) (2)

Equations (1) and (2) can be modified as follows:

f2=f0·cos(θ+φ) (3)

f1=f0·cos(θ−φ) (4)

If f1, f2, and φ are known, θ can be obtained by equations (5) and (6) given below:

tan θ={(f1+f2)/(f2−f1)}·tan θ (5)

θ=tan⁻¹{(f1+f2)/(f2−f1)}·tan θ (6)

In addition, f0 after angle correction can be obtained by equation (7):

f0=1/2{(f1+f2)²/cos²θ+(f2−f1)²/sin²θ}² (7)

When, therefore, actually applying the simultaneous multidirectional CWD function on a two-dimensional section, the apparatus executes transmission and reception by assigning frequencies, e.g., 1.8 MHz to one of a pair of deflected beams whose orientation directions (direction angles) are symmetrical about a central beam and 2.2 MHz to the other beam, with the central beam having a frequency of 2 MHz. This makes it possible to automatically estimate a true blood flow direction and the magnitude of the blood flow (velocity) and perform angle correction based on the Doppler shift velocities obtained from the respective orientation directions. In addition, using a plurality of pairs of frequencies can improve the estimation accuracy. For example, the apparatus executes the above calculations by using a plurality of pairs, for example, 1.9 MHz and 2.1 MHz, 1.8 MHz and 2.2 MHz, 1.7 MHz and 2.3 MHz, and 1.6 MHz and 2.4 MHz, and averages the calculation results. This makes it possible to implement angle correction with higher accuracy.

The above angle correction is three-dimensionally expanded. As shown in FIG. 19, for example, letting f1, f2, f3, and f4 be frequencies from points P1, P2, P3, and P4, the apparatus calculates projection vectors from the points P1 and P2 on a section (X-Z plane) in an azimuth direction and from the points P3 and P4 on a section (Y-Z plane) in an elevation direction by using a two-dimensional method. As a result, it is possible to acquire a correction angle θa and a correction velocity fa of the section in the azimuth direction and a correction angle θe and a correction velocity fe of the section in the elevation direction according to equations (8), (9), (10), and (11):

fa=1/2{(f1+f2)²/cos²φ+(f2−f1)²/sin²φ}² (8)

θa=tan⁻¹{(f1+f2)/(f2−f1)}·tan φ (9)

fe=1/2{(f4+f3)²/cos²φ+(f4−f3)²/sin²φ}² (10)

θe=tan⁻¹{(f4+f3)/(f4−f3)}·tan φ (11)

It is possible to obtain a three-dimensional angle correction f0 (absolute value) according to equations (12) and (13):

$$\begin{matrix}\vert f 0 \vert = {\{ {fe}^{2} + {( fa \cdot cos θ a )}^{2} \}}^{\frac{1}{2}} \\ = {\{ {fa}^{2} + {( fe \cdot cos θ e )}^{2} \}}^{\frac{1}{2}} ( 13 )\end{matrix} \qquad ( 12 )$$

When actually applying the simultaneous multidirectional CWD function to three-dimensional sections, the apparatus may execute transmission and reception by assigning different frequencies to a pair of deflected beams whose orientation directions (direction angles) are symmetrical about a central beam as in the case of two-dimensional sections. Using a plurality of pairs of two frequencies can improve the estimation accuracy as in the above case.

### Application Example 5

Application example 5 is designed to acquire the intravascular distribution of blood flow velocities by the simultaneous multidirectional CWD function using a two-dimensional ultrasonic probe.

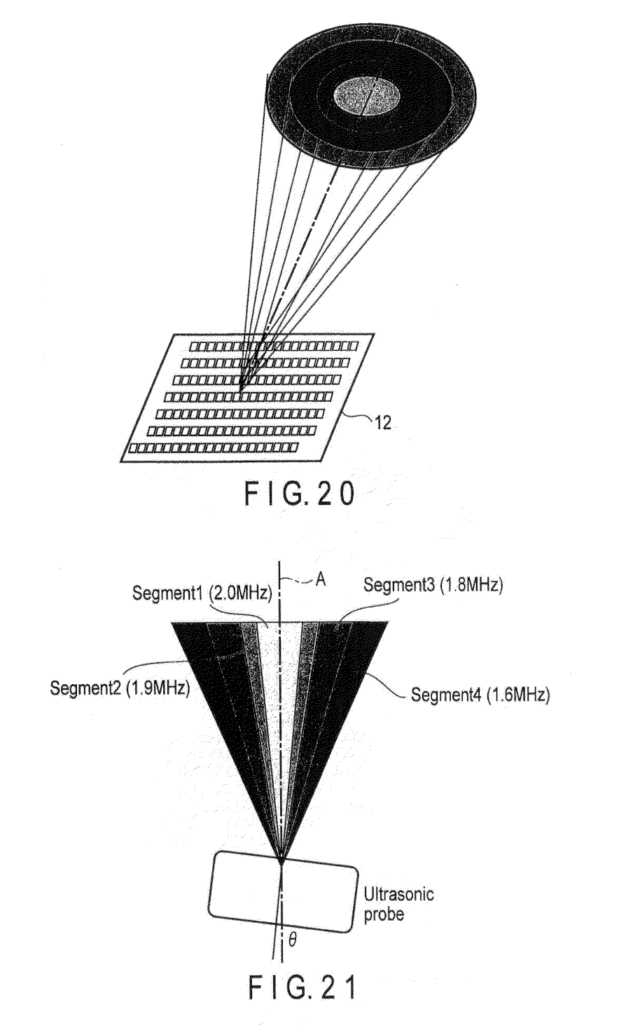

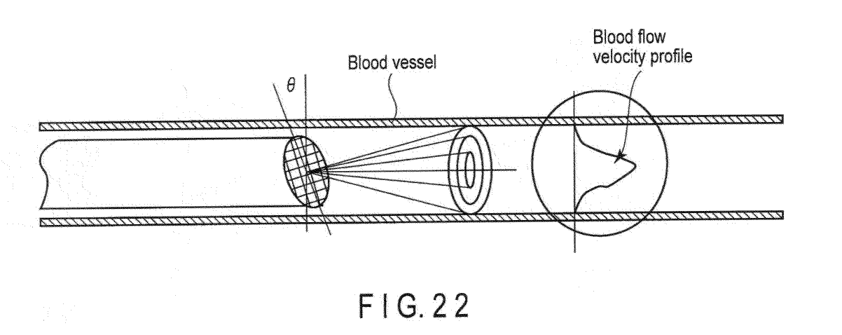

FIGS. 20, 21, and 22 are views for explaining application example 5 of the simultaneous multidirectional CWD function. As shown in FIG. 20, the apparatus executes ultrasonically scans, with the two-dimensional ultrasonic probe, a three-dimensional region (a three-dimensional region segmented like concentric cones) segmented into beams with the same frequency in a concentric form. For example, as shown in FIG. 21, the apparatus executes simultaneous multidirectional CWD upon respectively assigning 2.0 MHz, 1.9 MHz, 1.8 MHz, and 1.6 MHz to segments 1, 2, 3, and 4 in a concentric form including a central axis A. The apparatus can acquire a blood flow velocity and a power at each segment from the frequency distribution obtained for each segment. Mapping these pieces of information in correspondence with the spatial positions of these segments can estimate a three-dimensional intravascular distribution of blood flow velocities and the like. Applying this technique to an end-fire type angioscope, in particular, as shown in FIG. 22 can acquire a simple blood flow velocity profile in the blood vessel.

### Application Example 6

Application example 6 is designed to calculate a predetermined diagnostic index value such as a pulse wave velocity measurement by using the simultaneous multidirectional CWD function.

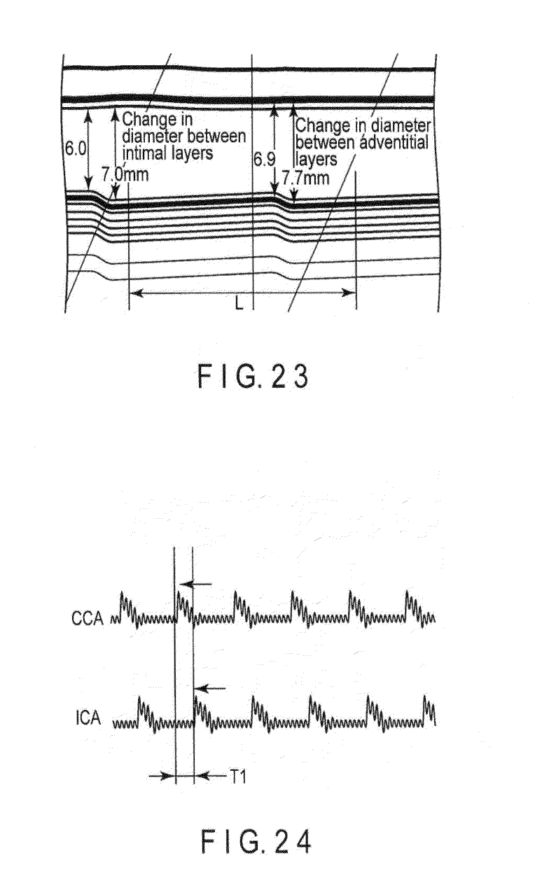

FIGS. 23, 24, and 25 are views for explaining application example 6 of the simultaneous multidirectional CWD function. For example, the apparatus executes the simultaneous multidirectional CWD function upon assigning different frequencies to two orientation directions to calculate a change in diameter between the intimal layers and a change in diameter between the adventitial layers from the Doppler images obtained in the respective orientation directions, as shown in FIG. 23. The apparatus then can calculate the inner diameter of the blood vessel from the calculation results. In addition, as shown in FIG. 24, the apparatus measures the maximum velocity of common carotid artery (CCA) obtained from a Doppler waveform in one orientation direction and the maximum velocity of internal carotid artery (ICA) obtained from a Doppler waveform in the other orientation direction, and obtains a peak time difference from the difference between the obtained CCA and ICA. The apparatus then can calculate a pulse wave (elastic wave of a blood vessel) velocity C from the distance between them. In addition, the apparatus can calculate an arteriosclerosis degree from the pulse wave velocity, the inner diameter of the blood vessel, and the like according to a predetermined formula.

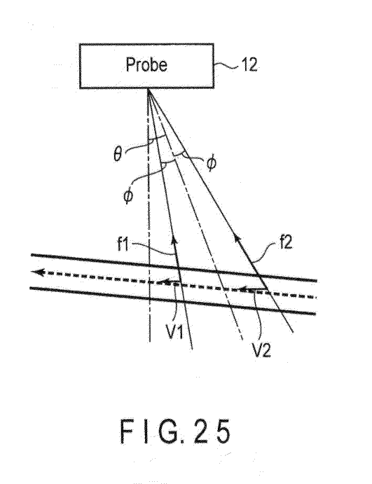

As shown in FIG. 25, let φ be the angle defined by the central beam and the blood vessel (blood flow) and φ be the angle defined by the central beams of a pair of reception beams. In this case, it is possible to estimate actual velocities V1 and V2 in the blood vessel from the geometric shapes of the beams and Doppler components (velocities) f1 and f2 of the observed beams. It is also possible to calculate a pulse wave velocity and a pressure loss originating from a pressure gradient from the estimated velocities V1 and V2.

### (Effects)

When performing blood flow measurement using the CWD method, this ultrasonic diagnostic apparatus can simultaneously execute CDW in the respective orientation directions by transmitting multiplex waves with different frequencies being assigned for the respective orientation directions of ultrasonic beams from the respective ultrasonic transducers and detecting the Doppler shift frequencies of the respective frequencies from the reflected waves obtained by the multiplex waves. The CDW method can therefore implement beam deflection equal to or more than that implemented by general phase delays, and can implement blood flow measurement in a wider range than the related art.

It is also possible to increase the S/N ratio by compounding echo signals obtained by multiplexing beam acoustic fields in the orientation directions.

It is also possible to grasp up to which orientation direction, for example, a reverse flow jet exerts influence (a quantitative distribution of a reverse flow jet) and the like from the maximum velocities and the power value for the respective frequencies assigned to the orientation directions and the distribution states of them in the beam array directions (orientation directions).

In addition, the apparatus executes the simultaneous multidirectional CWD function upon assigning different frequencies to a pair of deflected beams whose orientation directions (direction angles) are symmetrical about a central beam at a direction angle of θ. As a result, it is possible to automatically estimate a true blood flow direction and the magnitude of the blood flow and perform angle correction based on the Doppler shift velocities obtained from the respective orientation directions.

Furthermore, the apparatus executes simultaneous multidirectional CWD using a two-dimensional ultrasonic probe upon assigning different frequencies to the respective three-dimensional regions obtained by concentrically segmenting beam acoustic fields with the same frequency. It is possible to estimate a three-dimensional intravascular distribution of blood flow velocities or the like by acquiring a blood flow velocity and a power in each segment from a frequency distribution for each segment which is obtained as a result of the above operation and mapping these pieces of information in correspondence with the spatial positions of the respective segments.

Moreover, the apparatus executes the simultaneous multidirectional CWD function upon assigning different frequencies to two orientation directions, and calculates a change in diameter between the intimal layers and a change in diameter between the adventitial layers from the Doppler images obtained in the respective orientation directions. The apparatus then can calculate the inner diameter of the blood vessel from the calculation results. In addition, for example, the apparatus measures the maximum velocity of common carotid artery (CCA) obtained from a Doppler waveform in one orientation direction and the maximum velocity of internal carotid artery (ICA) obtained from a Doppler waveform in the other orientation direction, and obtains a peak time difference from the difference between the obtained CCA and ICA. The apparatus then can calculate a pulse wave (elastic wave of a blood vessel) velocity, the inner diameter of the blood vessel, arteriosclerosis degree, and the like from the distance between them.

### Second Embodiment

An ultrasonic diagnostic apparatus according to the second embodiment will be described next. The ultrasonic diagnostic apparatus according to the second embodiment includes a frequency-divided FMCWD function (to be described later). The arrangement of the ultrasonic diagnostic apparatus according to the second embodiment is nearly the same as that shown in FIG. 1 except for the functions of an ultrasonic transmission unit 21, ultrasonic reception unit 22, and control processor 28 and programs stored in a storage unit 29.

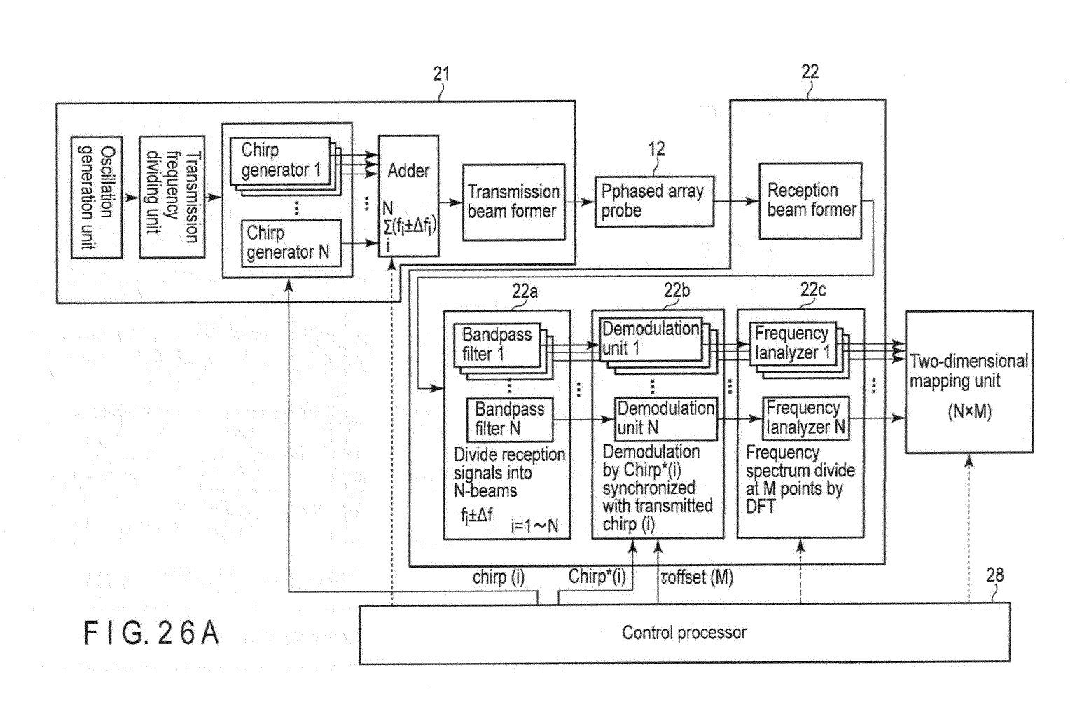

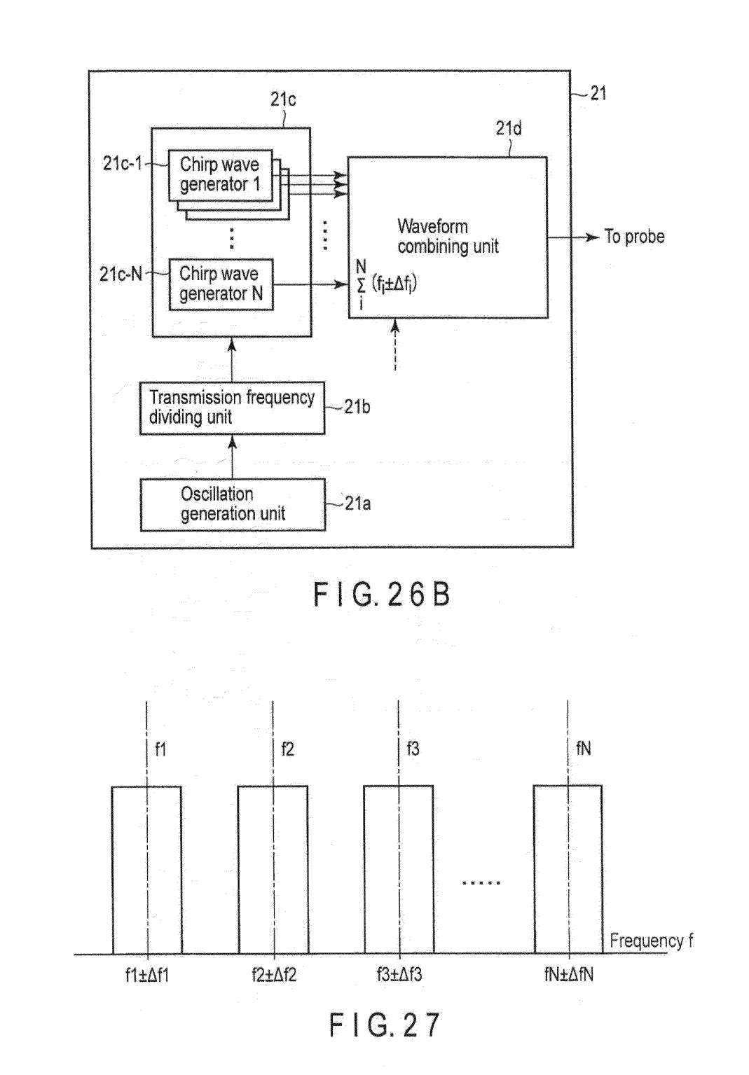

That is, as shown in FIG. 26A, the ultrasonic transmission unit 21 and the ultrasonic reception unit 22 execute transmission and reception for implementing the frequency-divided FMCWD function. Each function of the ultrasonic transmission unit 21 and the ultrasonic reception unit 22 are described later. The control processor 28 reads out a control program for implementing the frequency-divided FMCWD function (to be described later) from the storage unit 29, expands the program in its own memory, and executes control concerning simultaneous multidirectional CWD and calculations (calculations of a compound, the spatial distribution of signal intensities, automatic angle correction, the intravascular distribution of blood flow velocities, and diagnostic index values) using the signals in the respective orientation directions which are obtained by the simultaneous multidirectional CWD function. The storage unit 29 stores a control program for implementing the frequency-divided FMCWD function (to be described later).

### (Frequency-Divided FMCWD Function)

The frequency-divided FMCWD function of an ultrasonic diagnostic apparatus 1 will be described next. This function is a technique of implementing CWD exhibiting resolutions in both an orientation direction and a distance direction (depth direction). That is, when performing blood flow measurement using the CWD method, the apparatus transmits multiplex waves (multi-frequency transmission waves) with different fundamental frequencies assigned to the respective orientation directions of ultrasonic beams from the respective ultrasonic transducers while performing frequency modulation for each band. The apparatus detects the shift frequencies of the respective fundamental frequencies from the reflected waves obtained from the frequency-modulated multiplex waves to discriminate the reflected waves from the respective orientation directions and demodulate the discriminated reflected waves from the respective orientation directions, thereby achieving resolutions in the distance direction.

FIG. 26B shows the arrangement of the ultrasonic transmission unit 21 which implements the frequency-divided FMCWD function. The ultrasonic transmission unit 21 includes an oscillation generation unit 21a, a transmission frequency dividing unit 21b, a chirp wave generation unit 21c, and a waveform combining unit 21d.

The oscillation generation unit 21a repeatedly generates oscillation waveforms having a predetermined frequency fr Hz (period: 1/fr sec). In a 1/fr cycle, it is better that all the oscillation waveforms (chirp waveforms) are seamlessly repeated by each integer ratio. The transmission frequency dividing unit 21b frequency-divides oscillation waveforms to generate fundamental waveforms with different fundamental frequencies f1, f2, . . . , fN assigned in correspondence with orientation directions.

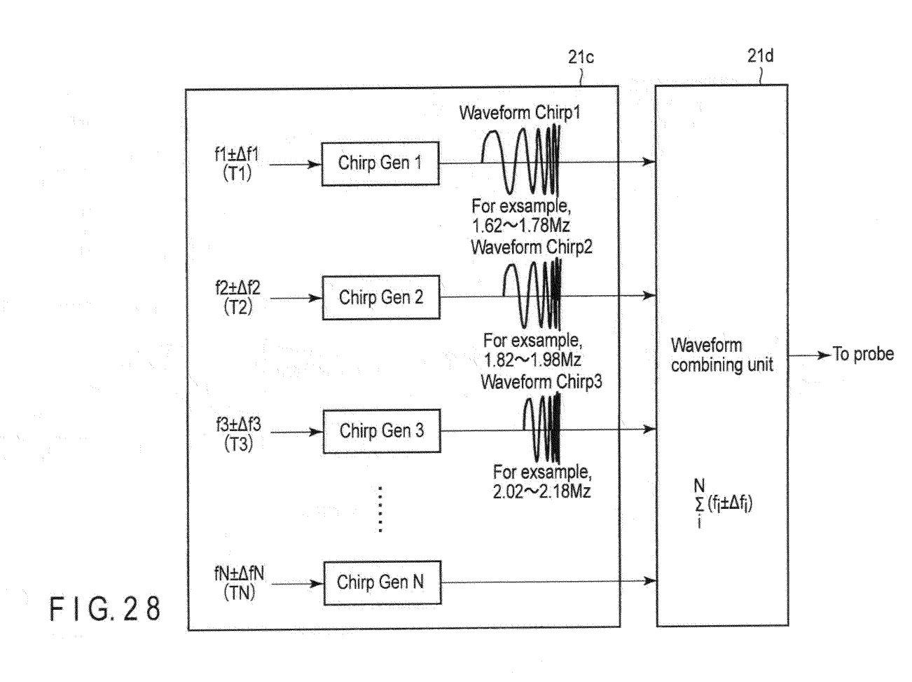

The chirp wave generation unit 21c includes chirp generators 21c-1 to 21c-N corresponding to the respective fundamental frequencies. The chirp generators 21c-1 to 21c-N sequentially input fundamental waveforms having corresponding fundamental frequencies from the transmission frequency dividing unit 21b. The chirp generators 21c-1 to 21c-N respectively generate chirp waves having bandwidths Δf1 to ΔfN with the fundamental frequencies f1, f2, . . . , fN being center frequencies based on the input fundamental waveforms. This makes chirp waves i having bands of fi±Δfi (where i is a natural number equal to or more than 2 and satisfying 1≦i≦N) perform band division, thereby ensuring N beams corresponding to N orientation directions, as shown in FIGS. 27 and 28.

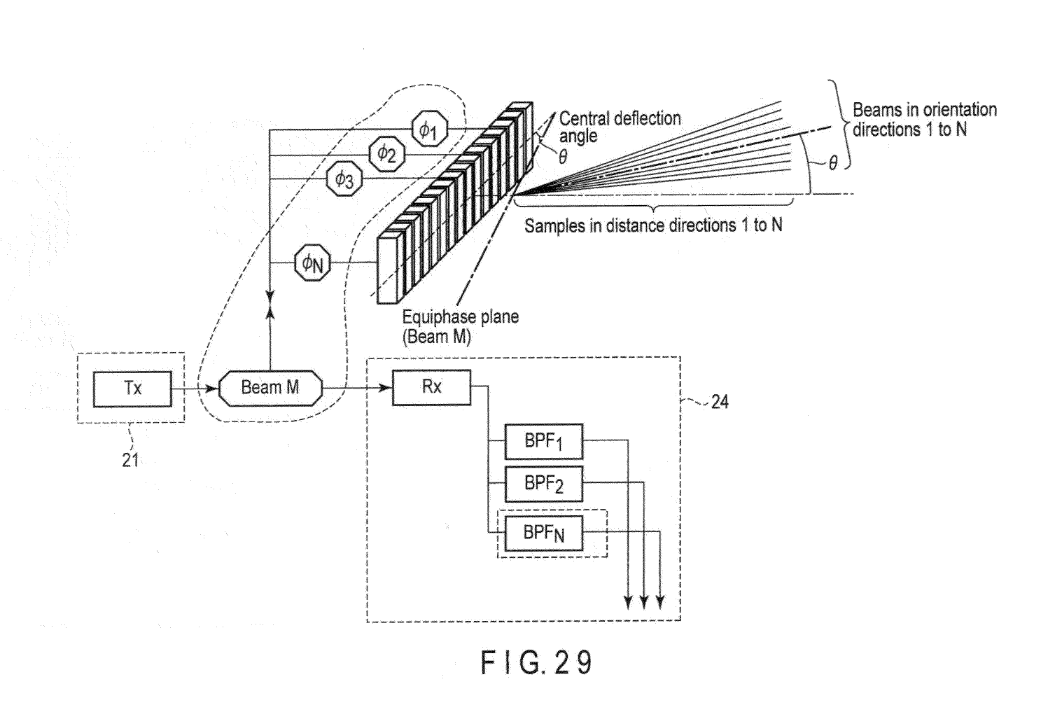

As shown in FIG. 28, the waveform combining unit 21d performs transmission beam forming by receiving and adding chirp waves from the chirp generators 21c-1 to 21c-N, and generates a multiplex transmission wave VM by multiplexing the respective chirp waves. The waveform combining unit 21d gives different phase delays φ₁, φ₂, φ₃, . . . , φ_{N} to the generated multiplex wave VM for the respective ultrasonic transducers, and supplies the resultant waves to the respective ultrasonic transducers. As a result, as shown in FIG. 29, the ultrasonic probe 12 continuously transmits transmission beams each obtained by multiplexing chirp wave 1 corresponding to an orientation direction θ1, chirp wave 2 corresponding to an orientation direction θ2, . . . , chirp wave N corresponding to an orientation direction θN. Note that FIG. 29 exemplifies the transmission beam with a central deflection angle θ.

The transmitted transmission beam is reflected by the inside of the object body and is received as a reflected wave by each ultrasonic transducer. The reception unit 22 executes reception processing based on the frequency-divided FMCWD function (to be described later) for each reflected wave received by a corresponding one of the ultrasonic transducers.

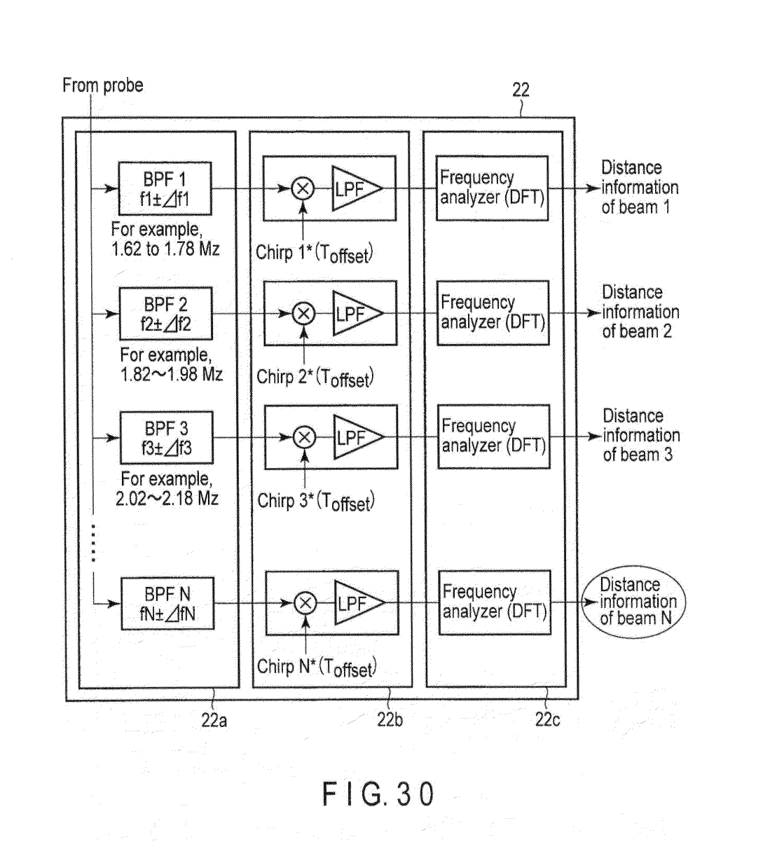

FIG. 30 is a block diagram showing the arrangement of the ultrasonic reception unit 22 which implements the frequency-divided FMCWD function. The ultrasonic reception unit 22 includes a bandpass filter array 22a, a demodulation unit 22b, and a frequency analysis unit 22c.

The bandpass filter array 22a includes bandpass filters 22a-1 to 22a-N corresponding to frequency bands f1±Δf1 to fN±ΔfN. The bandpass filters 22a-1 to 22a-N receive reception signals via the ultrasonic probe 12 and extract signals in the corresponding frequency bands respectively. This demultiplexes the signals into N chirp waves 1 to N respectively corresponding to the N orientation directions.

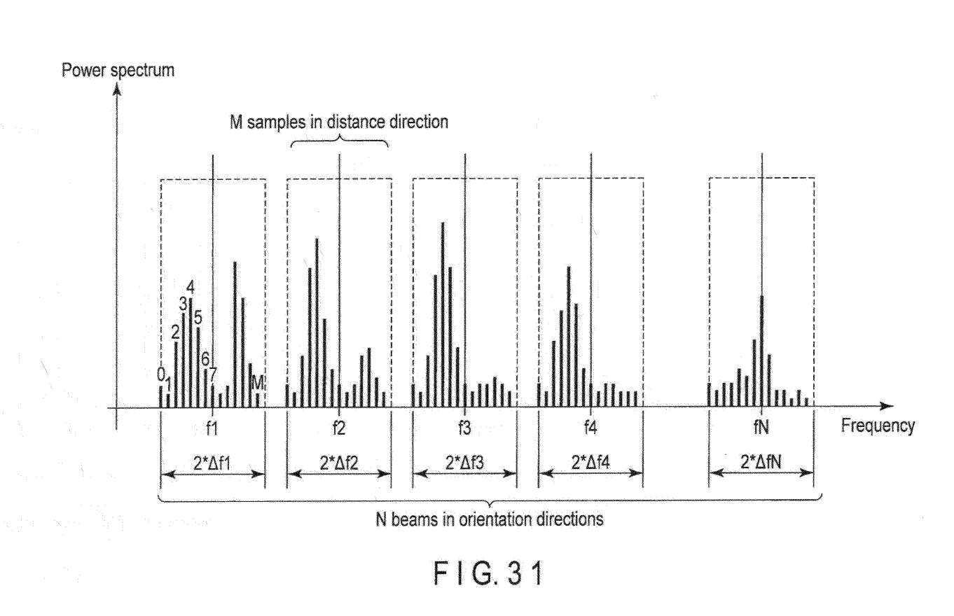

The demodulation unit 22b includes a plurality of demodulators 22b-1 to 22b-N corresponding to the respective frequency bands. The demodulators 22b-1 to 22b-N execute demodulation processing for chirp waves 1 to N having corresponding frequency bands. As shown in FIG. 31, this will detect the power spectra of N reception beams respectively corresponding to the N orientation directions band-divided into f1±Δf1 to fN±ΔfN.

The frequency analysis unit 22c includes N frequency analyzers 22c-1 to 22c-N corresponding to frequency bands f1±Δf1 to fN±ΔfN. The frequency analyzers 22c-1 to 22c-N transform frequency information into distance information by performing discrete Fourier transform of demodulated signals output from the demodulators 22b-1 to 22b-N. This detects distance information in the respective orientation directions (i.e., transmission/reception beams 1 to N).

The image generation unit 25 generates an ultrasonic image, in which pieces of information in the depths of the respective orientation directions are mapped, by using the distance information in each orientation direction. A display processing unit 27 performs predetermined display processing for the generated ultrasonic image. The monitor 14 then displays the resultant image in a predetermined form.

### Application Example

Each of the demodulators 22b-1 to 22b-N may execute any kind of demodulation processing. This application example will exemplify the demodulation processing of integrating the complex conjugate waveforms of chirp waves corresponding to the respective frequency bands, which are generated at the time of transmission, for chirp waves 1 to N output from the bandpass filters 22a-1 to 22a-N.

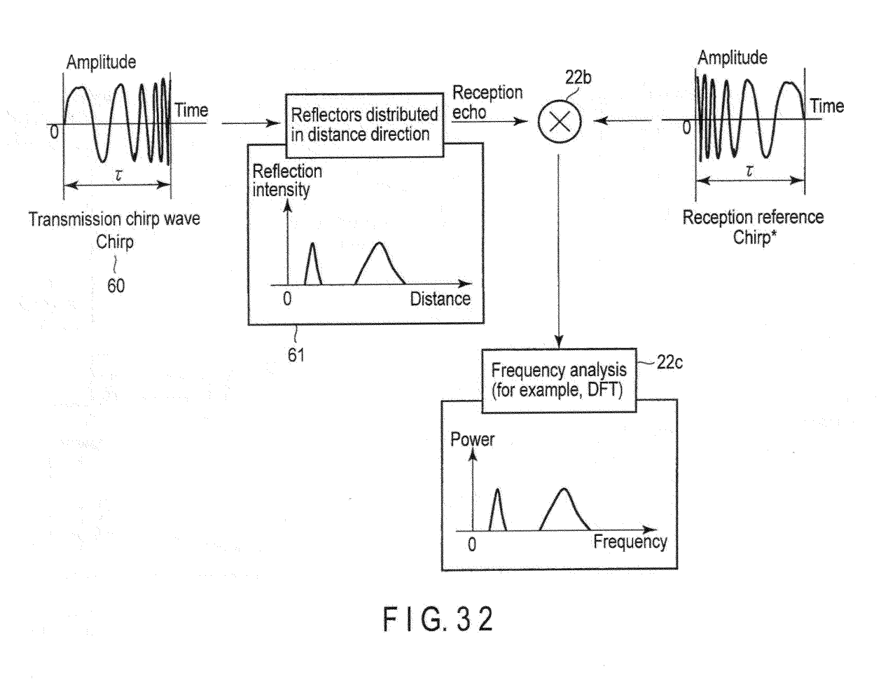

FIG. 32 is a conceptual view for explaining demodulation processing according to this application example. As shown in FIG. 32, in demodulation processing according to the application example, in accordance with an up (or down) modulation interval of each chirp wave 60 before combining in the waveform combining unit 21d, the demodulators 22b-1 to 22b-N of the demodulation unit 22b, which correspond to the respective frequency bands, each perform demodulation by integrating reception reference chirp wave 63 (that is, the complex conjugate waveform of chirp wave 60) oriented in an opposite direction (i.e., the down direction if the chirp wave before combining is in the up direction, and vice versa) corresponding to a distance direction observation interval converted from an ultrasonic propagation velocity.

In general, one chirp transmission 60 (the up or down direction) and a detection output from a single reception detector (τ=0) include all reflector intensity information 61 in the corresponding distance direction observation interval. It is possible to detect a distance direction reflection intensity distribution as a frequency spectrum by performing one frequency analysis (DFT: Discrete Fourier Transform) or the like for all observation intervals by using the detection output. This signal processing makes it possible to greatly reduce the scale of hardware/software. At the same time, this technique obtains a pulse compression effect, and hence involves less waveform tailing and the like than the pulse method. This makes it possible to achieve good distance resolution.

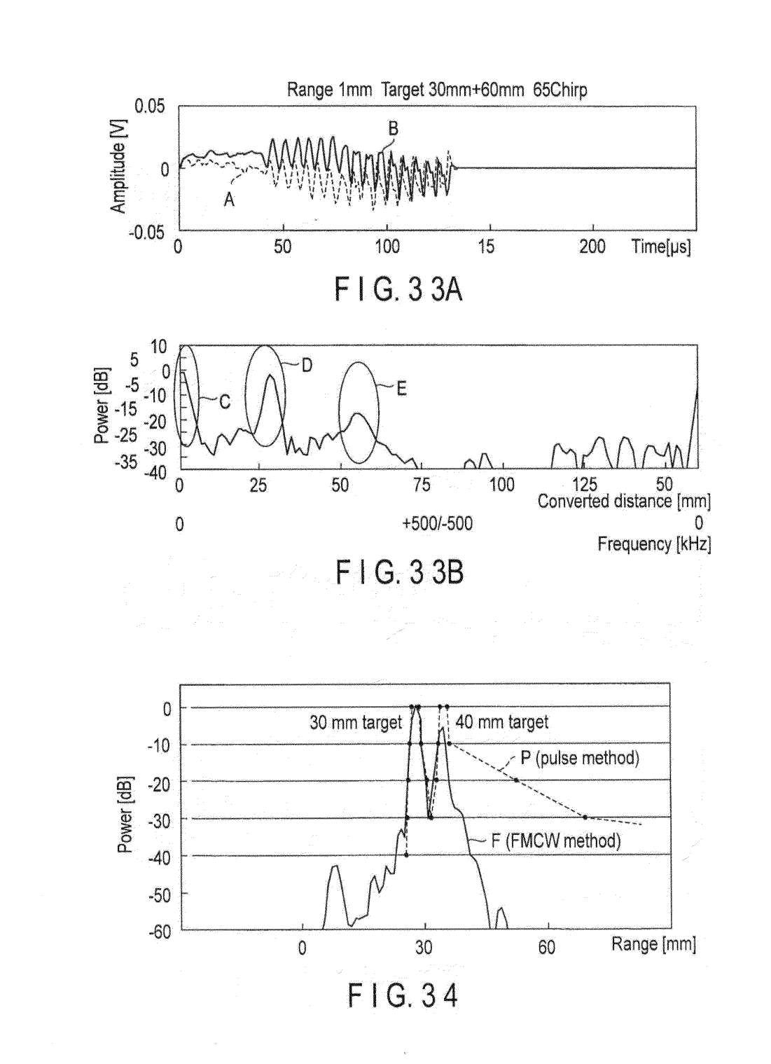

FIG. 33A shows a process in a distance direction with fixing an orientation direction and the waveform obtained by demodulating the reflection signals from pin targets at the positions corresponding to 30 mm and 60 mm with a multiphase demodulation range being fixed to 0 mm. The unit on the abscissa (time axis) is 1 μs, and the unit on the ordinate is 0.1 Vpp at full swing.

Note that since a reception wave has undergone complex demodulation, a waveform A is an I-phase signal, and a waveform B is a Q-phase signal. FIG. 33B shows spectra with different depths in the range of ±500 kHz, which are obtained by applying a Hamming window to the result obtained by frequency-analyzing the waveform in FIG. 33A, calculating a power spectrum after 128-point FFT, and logarithmically compressing the resultant information. That is, a spectrum C is a component originating from reflection by the probe surface (body surface 0 mm), a spectrum D is a reflection component from a pin target at the position corresponding to 30 mm, and a spectrum E is a reflection component from the pin target at the position corresponding to 60 mm. The ordinate corresponds to powers (dB). On the abscissa, FFT outputs are not rearranged/corrected, and hence the frequency is 0 Hz at the left end, and increases toward the middle. The frequency is 500 kHz at the middle position, becomes −500 kHz on the right half portion from the middle, decreases in negative absolute value, and becomes 0 Hz at the right end.

FIG. 34 shows the result obtained by generating a comparative profile from a B-mode image complying with the spectrum shown in FIG. 33A and matching the peak position reference with the A mode of FMCW (reducing the band to ½, while keeping the number of FFT points unchanged, to improve the sensitivity. Image signals of pin targets at 30 mm and 40 mm underwater are obtained by logarithmically compressing reflection echo powers, obtained by the general pulse method, for display by STC correction (gain correction in accordance with distances). In contrast, although no STC correction is applied to a spectrum F obtained by logarithmically compressing the reflection echo power by the FMCW method, the pin target at the 30 mm position is properly isolated from the pin target at the 40 mm position. It can be recognized from FIG. 34 that the FMCW method is free from waveform tailing behind a solid body due to the pulse compression effect, and high distance resolution is achieved in spite of the use of a continuous wave pencil probe (2 MHz).

This embodiment has been described by using analog circuits (BPF and the like) with reference to FIGS. 26, 28, 29, and 30. If, however, a D/A converter and an A/D converter which convert analog and digital signals have sufficiently high sampling frequencies, it is possible to perform waveform multiplexing/demultiplexing by using digital processing and software.

### (Effects)

When performing blood flow measurement by the CWD method, the ultrasonic diagnostic apparatus transmits multi-frequency transmission waves with different fundamental frequencies assigned to the respective orientation directions of ultrasonic beams from the respectively ultrasonic transducers, while performing frequency modulation for the respective bands. In addition, the apparatus detects the shift frequencies of the respective fundamental frequencies from the reflected waves obtained by the frequency-modulated multi-frequency transmission waves to discriminate the reflected waves from the respective orientation directions and demodulate the discriminated reflected waves for the respective orientation directions, thereby converting frequency information into distance information. This makes it possible to acquire information at each depth (i.e., depth range 1 to M) in each orientation direction (i.e., for each of transmission/reception beams 1 to N) by using the CDW method as well.

Note that the present invention is not limited to the embodiment described above, and constituent elements can be modified and embodied in the execution stage within the spirit and scope of the invention.

Each function associated with this embodiment can also be implemented by installing programs for executing control on the functions in a computer such as a workstation and expanding them in a memory. In this case, the programs which can cause the computer to execute the corresponding techniques can be distributed by being stored in recording media such as magnetic disks (Floppy® disks, hard disks, and the like), optical disks (CD-ROMs, DVDs, and the like), and semiconductor memories.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a transmission unit configured to continuously generate driving signals by frequency-modulating a plurality of waveforms having a plurality of center frequencies respectively assigned to a plurality of orientation directions and multiplexing the plurality of waveforms and transmit continuous waves deflected from a perpendicular direction to an array plane of ultrasonic transducers of an ultrasonic probe via an ultrasonic probe by supplying the driving signals to the ultrasonic transducers with different delay times;
a reception unit configured to generate a plurality of beam signals corresponding to the respective orientation directions by adding the respective echo signals received by the respective ultrasonic transducers with different delay times for the respective ultrasonic transducers and demultiplexing the signals for the respective center frequencies and demodulate a plurality of beam signals corresponding to the respective orientation directions, frequency-analyze the plurality of demodulated beam signals, and calculate beam signals including distance information concerning a depth direction in each orientation direction;
a frequency analyzing unit configured to detect shift frequency spectrums for the respective orientation directions by using a plurality of beam signals including distance information concerning the respective orientation directions; and
an image generation unit configured to generate an ultrasonic image based on the shift frequency spectrums concerning the depth direction in the each orientation direction.

2. The apparatus of claim 1, wherein the reception unit extracts a plurality of beam signals corresponding to the plurality of orientation directions by using a bandpass filter provided for the each center frequency corresponding to a bandwidth concerning the frequency modulation.

3. The apparatus of claim 1, wherein the reception unit demodulates the plurality of beam signals by using complex conjugate waveforms of the plurality of waveforms.

4. The apparatus of claim 1, wherein the reception unit performs demodulation of complex-conjugate to the transmitted waveforms corresponding to a distance direction observation interval converted from an ultrasonic propagation velocity in accordance with a modulation interval of a plurality of waveforms, and
the image generation unit generates the ultrasonic image in which a frequency distribution of spectra obtained by frequency analysis corresponding to all observation intervals corresponds to a distance direction reflection intensity distribution.

5. The apparatus of claim 1, wherein the transmission unit executes beam steering concerning the respective orientation directions by phase-delaying the respective driving signals supplied to the plurality of ultrasonic transducers.

6. The apparatus of claim 1, wherein the transmission unit spatially multiplexes ultrasonic waves having a plurality of center frequencies respectively and transmitted from the respective ultrasonic transducers in response to the driving signals, and
the reception unit generates the plurality of beam signals corresponding to the respective orientation directions by determining frequency bands of echo signals based on the ultrasonic waves having a plurality of center frequencies respectively.

7. The apparatus of claim 1, wherein the frequency analyzing unit calculates a distribution of measurement values concerning the respective orientation directions based on shift frequencies for the respective orientation directions.

8. The apparatus of claim 1, wherein the transmission unit assigns different frequencies to two orientation directions symmetrical about a central beam at an arbitrary direction angle of θ, and
the frequency analyzing unit estimates at least one of a blood flow direction and a magnitude of the blood flow in the object based on shift velocities obtained from the two symmetrical orientation directions.

9. The apparatus of claim 1, wherein the transmission unit assigns different frequencies to two orientation directions symmetrical about a central beam at an arbitrary direction angle of θ, and
the frequency analyzing unit corrects an angle of the central beam based on shift velocities obtained from the two symmetrical orientation directions.

10. The apparatus of claim 1, wherein the ultrasonic probe comprises a two-dimensional probe having the plurality of ultrasonic transducers arrayed two-dimensionally, and
the transmission unit supplies the driving signals corresponding to predetermined frequencies to the plurality of ultrasonic transducers so as to form a three-dimensional acoustic field obtained by concentrically segmenting a transmission ultrasonic acoustic field of the same frequency.

11. The apparatus of claim 1, further comprising a calculation unit configured to calculate a predetermined diagnostic index by using shift frequencies for the respective orientation directions.

12. An ultrasonic diagnostic apparatus comprising:
a transmission unit configured to continuously generate driving signals by frequency-modulating a plurality of waveforms having a plurality of center frequencies respectively assigned to a plurality of orientation directions and multiplexing the plurality of waveforms and transmit continuous waves deflected from a perpendicular direction to an array plane of ultrasonic transducers of an ultrasonic probe via an ultrasonic probe by supplying the driving signals to the ultrasonic transducers with different delay times;
a reception unit configured to generate a plurality of beam signals corresponding to the respective orientation directions by adding the respective echo signals received by the respective ultrasonic transducers with different delay times for the respective ultrasonic transducers and demultiplexing the signals for the respective center frequencies; and
a frequency analyzing unit configured to detect shift frequencies for the respective orientation directions by using a plurality of beam signals corresponding to the respective orientation directions.

13. The apparatus of claim 12, wherein the transmission unit executes beam steering concerning the respective orientation directions by phase-delaying the respective driving signals supplied to the plurality of ultrasonic transducers.

14. The apparatus of claim 12, wherein the transmission unit spatially multiplexes ultrasonic waves having a plurality of center frequencies respectively and transmitted from the respective ultrasonic transducers in response to the driving signals, and
the reception unit generates the plurality of beam signals corresponding to the respective orientation directions by determining frequency bands of echo signals based on the ultrasonic waves having a plurality of center frequencies respectively.

15. The apparatus of claim 12, wherein the frequency analyzing unit calculates a distribution of measurement values concerning the respective orientation directions based on shift frequencies for the respective orientation directions.

16. The apparatus of claim 12, wherein the transmission unit assigns different frequencies to two orientation directions symmetrical about a central beam at an arbitrary direction angle of θ, and
the frequency analyzing unit estimates at least one of a blood flow direction and a magnitude of the blood flow in the object based on shift velocities obtained from the two symmetrical orientation directions.

17. The apparatus of claim 12, wherein the transmission unit assigns different frequencies to two orientation directions symmetrical about a central beam at an arbitrary direction angle of θ, and
the frequency analyzing unit corrects an angle of the central beam based on shift velocities obtained from the two symmetrical orientation directions.

18. The apparatus of claim 12, wherein the ultrasonic probe comprises a two-dimensional probe having the plurality of ultrasonic transducers arrayed two-dimensionally, and
the transmission unit supplies the driving signals corresponding to predetermined frequencies to the plurality of ultrasonic transducers so as to form a three-dimensional acoustic field obtained by concentrically segmenting a transmission ultrasonic acoustic field of the same frequency.

19. The apparatus of claim 12, further comprising a calculation unit configured to calculate a predetermined diagnostic index by using shift frequencies for the respective orientation directions.

20. An ultrasonic diagnostic apparatus control method comprising:
generating driving signals continuously by frequency-modulating a plurality of waveforms having a plurality of center frequencies respectively assigned to a plurality of orientation directions and multiplexing the plurality of waveforms;
transmitting continuous waves deflected from a perpendicular direction to an array plane of ultrasonic transducers of an ultrasonic probe via an ultrasonic probe by supplying the driving signals to the ultrasonic transducers with different delay times;
generating a plurality of beam signals corresponding to the respective orientation directions by adding the respective echo signals received by the respective ultrasonic transducers with different delay times for the respective ultrasonic transducers and demultiplexing the signals for the respective center frequencies;
demodulating a plurality of beam signals corresponding to the respective orientation directions, frequency-analyzes the plurality of demodulated beam signals;
calculating beam signals including distance information concerning a depth direction in each orientation direction;
detecting shift frequency spectrums for the respective orientation directions by using a plurality of beam signals including distance information concerning the respective orientation directions; and
generating an ultrasonic image based on the shift frequency spectrums concerning the depth direction in the each orientation direction.

21. An ultrasonic diagnostic apparatus control method comprising:
generating driving signals continuously by frequency-modulating a plurality of waveforms having a plurality of center frequencies respectively assigned to a plurality of orientation directions and multiplexing the plurality of waveforms;
transmitting continuous waves deflected from a perpendicular direction to an array plane of ultrasonic transducers of an ultrasonic probe via an ultrasonic probe by supplying the driving signals to the ultrasonic transducers with different delay times;
generating a plurality of beam signals corresponding to the respective orientation directions by adding the respective echo signals received by the respective ultrasonic transducers with different delay times for the respective ultrasonic transducers and demultiplexing the signals for the respective center frequencies; and
detecting shift frequencies for the respective orientation directions by using a plurality of beam signals corresponding to the respective orientation directions.
